(19) United States
(12) Patent Application Publication
(10) Pub. No.: US 2014/0336759 A1
(43) Pub. Date: Nov. 13, 2014

(54) IMPLANTABLE NIPPLE AND AREOLA PROSTHESIS

(71) Applicant: Patricia G. Martin, Tuscon, AZ (US)

(72) Inventor: Patricia G. Martin, Tuscon, AZ (US)

(21) Appl. No.: 13/889,319

(22) Filed: May 7, 2013

(51) Int. Cl. A61F 2/12

(52) U.S. Cl. CPC A61F 2/12
USPC 623/8

(57) ABSTRACT

An implantable nipple and areola prosthesis for post-mastectomy breast cancer patients includes a conical areola position bearing a matrix of suture holes and a protruding nipple portion. During post-mastectomy breast reconstruction surgery, a surgeon implants the prosthesis under the pectoralis major muscle, placing the implant with the nipple prominence anterior and areola portion posterior. The prosthesis is fixed with dissolving sutures in a circumferential fashion through suture holes in the areola portion of the implant. Scar tissue stabilizes the implant and once healed, cosmetic tattooing may be performed to create a realistic areola and nipple.

This application claims the benefit of the filing date of provisional application No. 61/644,344, filed on May 8, 2012.

### BACKGROUND

The invention relates to an implantable nipple and areola prosthesis for post-mastectomy breast cancer patients, and method thereof.

Known is U.S. Pat. No. 6,464,726 issue to Heljenek on Oct. 15, 2002. Heljenek discloses a breast implant system and method of augmentation. The system includes two separate breast prostheses that are used to enhance each single breast. A first breast prosthesis is surgically implanted under the pectoral muscle behind the breast. The first breast prosthesis augments the posterior section of the breast nearest the chest. A second breast prosthesis is provided that is surgically implanted between the breast tissue and the pectoral muscle. The second breast prosthesis augments the anterior section of the breast in front of the pectoral muscle. The second breast prosthesis provides the breast with a large size and soft textures, while the first breast prosthesis helps blend the augmented breast into the chest for a more natural appearance.

Also known is U.S. Pat. No. 5,824,076 issued to Knowlton on Oct. 20, 1998. Knowlton discloses a surgical method for breast reconstruction using a neovascular tissue peg. The method comprises the steps of: placing a sub muscular breast expander beneath a muscle; expanding the breast for a period of time that permits neovascular connections to grow between the muscle and cutaneous tissue which overlays the muscle; and, surgically reconstructing a nipple from the neovascularized cutaneous tissue overlaying the muscle, whereby the expanding step directly contributes an amount of tissue that permits achievement of the breast reconstruction. The step of recreating the nipple areolar complex can comprise performing a bowtie reconstruction technique. Typically, the expander is placed beneath a pectoralis major muscle.

Finally, U.S. Pat. No. 4,778,465 issued to Wilkins on Oct. 18, 1988 discloses a surgically implantable areola and nipple prosthesis. The prosthesis comprises a base and a nipple forming protrusions, wherein said base is concave on one surface and convex on the other surface and wherein the nipple forming protrusion is integrally formed onto the convex surface, and wherein the concave surface of the implantable nipple prosthesis may be affixed to a usual breast implantable sac in a location and manner so that the nipple forming prosthesis is oriented in as nearly normal position as possible, or wherein the invention may be implanted in a pocket in tissue as a separate item; and wherein the skin and tissue of the patient is closed about the areola and nipple forming prosthesis, and wherein the patient's skin will naturally assume the shape of the areola and nipple forming prosthesis, and wherein the exterior skin now shaped as an areola and nipple as be colored to resemble an areola and nipple, and wherein the coloring may be of the removable type, such as make-up, or of the more permanent type, such as dyeing or tattooing.

One major difference between the present invention and the prior art is that the present invention already incorporate a breast implant, and comprises one piece. It is not attached to a breast implant as in Wilkins, nor come in two separate pieces as in Heljenek. Moreover, the proposed invention has peripheral holes for suturing the implant.

Furthermore, the method of the present invention differs in that it comprises the following steps: 1. Reopen mastectomy scar; 2. Develop the plane beneath the pectoralis muscle; 3. Decide where the prominence of the nipple should be so that it is at least 1.5 to 2 cm from the incision line; 4. Pass the suture though one of the peripheral holes in the implant from the undersurface to the front; 5. Pass the suture through the undersurface of the muscle; 6. The suture goes back to the front of the prosthesis to the undersurface of the implants; 7. This may have to be done several times to get the suture to pull the prosthesis to the proper position; 8. At least four sutures should be placed in a similar fashion. With each succeeding placement of the sutures, less positioning is required.

### SUMMARY

The invention is an implantable nipple and areola prosthesis for post-mastectomy breast cancer patients, and a method of use. The system is designed as a fully adjustable and malleable areolar prosthesis, and is preferably formed from a silicone elastomer. During post-mastectomy breast reconstruction surgery, a surgeon implants the prosthesis under the pectoralis major muscle. The implant is placed with the nipple prominence anterior and mesh portion posterior. The prosthesis is fixed with dissolving sutures in a circumferential fashion through the mesh portion of the implant. Scar tissue stabilizes the implant and when the suture is well healed, cosmetic tattooing may be performed. The implant may also be made of alloplastic materials. By installing the prosthesis during post-mastectomy breast reconstruction surgery, the method is a minimally-invasive procedure and also provides a long-lasting projection and overall patient satisfaction.

### BRIEF DESCRIPTION OF THE FIGURES

FIG. 1 is a side view of a prosthetic areolar implant having a nipple portion.

FIG. 2 is a top view of it prosthetic areolar implant having a nipple portion.

FIG. 3 is a perspective view of a prosthetic areolar implant being sutured in position.

### REFERENCE NUMBERS

10 implant
12 areola portion
14 nipple portion
16 suture holes
18 peripheral edge
20 outer concentric ring
22 inner concentric ring
24 mastectomy scar incision line
26 pectoralis major muscle
28 subdermal tissue
30 suture

### DESCRIPTION

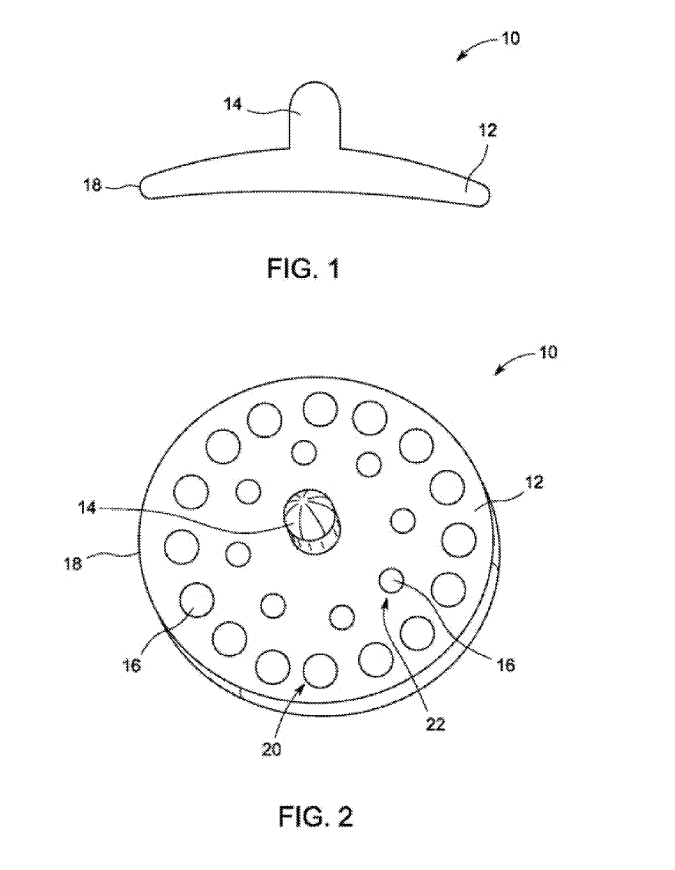

Referring to FIG. 1, a prosthetic areolar implant 10 is shown in side view. The implant 10 comprises an areola portion 12 and a nipple portion 14. In preferred embodiments, the implant 10 may be made of a silicon elastomer, or from alloplastic materials. In any event, the material comprising the implant 10 should be resilient enough to from a protrusion through a user's skin sufficient to mimic a typical human breast nipple. To accomplish these ends, the nipple portion 14 may be made in a variety of shapes and thicknesses, depending on the nipple shaped desired in the post-mastectomy breast region, or to match an existing nipple in the event of a single mastectomy.

Referring to FIG. 2, in plan view, the prosthetic areolar implant 10 comprises suture holes 16 in addition to the areola portion 12 and nipple portion 14. The suture holes 16 are ideally placed adjacent the peripheral edge 18 of the implant 10 to facilitate placement and resilient positioning of the implant 10 during surgery. Preferably a plurality of suture holes 16 are disposed on the areola portion 12 of the implant 10. In one embodiment the suture holes 16 may extend around the peripheral edge 18 in a ring. In another preferred embodiment, additional suture holes 16 may be incorporated into the areola portion 12 such that concentric rings are formed, including an outer concentric ring 20 and an inner concentric ring 22. In another embodiment, the suture holes 16 of the inner concentric ring 22 are smaller than the suture holes 16 of the outer concentric ring 20.

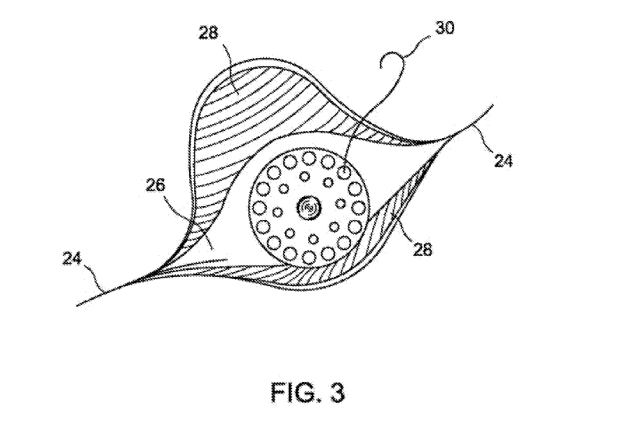

Referring to FIG. 3, the areolar implant 10 is shown in use during implantation. In order to install the implant, a patient's mastectomy scar incision line 24 is reopened, and a plane beneath a pectoralis major muscle 26 is developed by separating the pectoralis major muscle 26 from the subdermal tissue 28 which may comprise scar tissue or a capsule over the breast implant. Ideally, the areolar implant 10 is positioned wherein the prominence of the patient's nipple should be, and so that it is at least 1.5 to 2 cm from the mastectomy scar incision line 24.

Still referring to FIG. 3, the surgeon passes the suture 30 through the suture holes 16 of the areola portion 12. Preferably, the suture 30 will be passed through a suture hole 16 from the posterior surface to the anterior surface, including passing the suture 30 through posterior surface to the anterior surface of the pectoralis major muscle 26. The suture 30 may go back to the front of the implant 10 from the undersurface of the implant 10, and this process may have to be done several times to get the suture 30 to pull the implant 10 to the proper position. In one preferred embodiment, at least four sutures 30 should be placed in a similar fashion. With each succeeding placement of the sutures, less positioning is required.

While the apparatus has been described in detail with reference to specific embodiments thereof, it will be apparent to one skilled in the art that various changes and modifications can be made therein without departing from the spirit and scope thereof. Thus, it is intended that the present description cover the modifications and variations of the apparatus provided they come within the scope of the appended claims and their equivalents.

What is claimed is:

1. An areola and implant comprising:
a generally conical areola portion having an inner concave surface conforming to the shape of a human breast, and an outer convex surface;
the areola portion having suture holes in the areola portion for suturing the areola portion to the pectoralis major muscle; and
a projecting portion at the apex of the areola portion corresponding in shape to a human nipple.

2. The implant of claim 1 wherein the suture holes are disposed along the periphery of the areola portion.

3. The implant of claim 1 further comprising two concentric rings of suture holes.

4. The implant of claim 3 wherein the suture holes on an inner ring are smaller than the suture holes on an outer ring.

5. The implant of claim 1 further comprising an areola portion and a nipple portion comprising silicon elastomer.

6. A method of implanting an areola and nipple prosthesis, comprising the steps of:
reopening a mastectomy scar having an incision line;
developing a plane beneath a pectoralis major muscle by separating the pectoralis major muscle from subdermal tissue;
identifying a location of prominence for a nipple at least 1.5 to 2 cm from the incision line;
placing an areolar prosthesis, having an areolar portion with peripheral holes and a nipple portion, between the pectoralis major muscle and subdermal tissue such that the nipple portion is disposed at the location of prominence; and
passing a suture through one of peripheral holes and the pectoralis major muscle.

7. The method of claim 6 further comprising the step of adjusting the position of the areolar implant during suturing.

8. The method of claim 6 further comprising the step of passing the suture from the undersurface of the areolar portion to the front.

9. The method of claim 6 further comprising the step of passing the from the undersurface of the pectoralis major muscle to the front.

10. The method of claim 6 further comprising the step of suturing multiple peripheral holes.

11. The method of claim 6 wherein the subdermal tissue is scar tissue.
